(19) United States
(12) Patent Application Publication
(10) Pub. No.: US 2025/0176929 A1
(43) Pub. Date: Jun. 5, 2025

(54) PROVISION OF A RADIATION EXPOSURE DISTRIBUTION FOR AN IMAGING SYSTEM WITH A RADIATION SOURCE

(71) Applicant: Siemens Healthineers AG, Forchheim (DE)

(72) Inventor: Annette Birkhold, Stuttgart (DE)

(21) Appl. No.: 18/962,085

(22) Filed: Nov. 27, 2024

(30) Foreign Application Priority Data
Nov. 30, 2023 (DE) 10 2023 212 010.0

(51) Int. Cl. A61B 6/00

(52) U.S. Cl. CPC A61B 6/542; A61B 6/545

(57) ABSTRACT

A computer-implemented method for more accurately providing exposure distribution data for radiation from a radiation source of an imaging system, the method comprising receiving setting data of the imaging system, receiving a radiation exposure parameter associated with the setting data, receiving data associated with the setting data and relating to an intensity distribution of the radiation on a radiation detector of the imaging system, applying a first trained function to the setting data, the exposure parameter and the intensity distribution data as first input data for the first trained function, the exposure distribution data being generated as first output data from the first trained function, providing the exposure distribution data; the intensity distribution data being provided by a second trained function being applied to the setting data as second input data for the second trained function, and the intensity distribution data being generated as second output data from the second trained function.

### CROSS REFERENCE TO RELATED APPLICATIONS

This application claims the benefit of DE 10 2023 212 010.0 filed on Nov. 30, 2023, which is hereby incorporated by reference in its entirety.

### FIELD

Embodiments relate to a computer-implemented method for providing exposure distribution data for radiation from a radiation source of an imaging system.

### BACKGROUND

In imaging systems with a radiation source for producing ionizing radiation, it is desirable, for example for regulatory reasons, to document radiation exposure for patients and medical personnel as accurately as possible. A corresponding radiation exposure parameter may to this end be measured during operation of the imaging system.

In X-ray-based imaging systems, such as X-ray-based angiography systems, a dose area product (DAP) may to this end, for example, be measured as the radiation exposure parameter in the imaging system's beam path using a measuring device known as a DAP chamber. Apart from for the stated regulatory requirements, the measured DAP may also be used for closed-loop control of the radiation source.

Such a DAP is, however, only capable of providing a summed exposure over the irradiated area, but not a distribution of the exposure over this area. For example, it cannot be assumed therefrom that exposure is identically, i.e., homogeneously, distributed over the area; instead, radiation exposure may be non-homogeneously distributed thereover. For example, the radiation source emits non-homogeneously distributed radiation over the area, that may result in a non-homogeneous exposure distribution of this radiation over the irradiated area. It is thus also incorrect to estimate the local exposure to radiation that may occur on the skin, vessels, organs or the like, for instance in X-ray-based angiography systems, solely on the basis of the DAP. Local exposure may instead be higher than the hypothetically homogeneously distributed exposure on the basis of the DAP.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments accurately determine the radiation exposure distribution from a radiation source of an imaging system.

Embodiments are based on the concept of generating, at least using a trained function, exposure distribution data as first output data on the basis of setting data of the imaging system, the exposure parameter and data relating to the intensity distribution of the radiation on a radiation detector.

One aspect provides a computer-implemented method for providing exposure distribution data for radiation from a radiation source, for example a radiation source for generating ionizing radiation, of an imaging system.

The exposure distribution may also be designated dose distribution. For example, the exposure distribution data may take the form of image data that maps the distribution of exposure on a two-dimensional area. A pixel value of the image may here indicate the local exposure to radiation at the pixel.

Setting data of the imaging system is received in one step of the computer-implemented method. An exposure parameter or dose parameter of the radiation and associated with the setting data is received in a further step. Data associated with the setting data and relating to an intensity distribution of the radiation on a radiation detector of the imaging system is received in a further step.

In a further step, a first trained function is applied to the received setting data, the received exposure parameter and the received intensity distribution data as first input data for the first function. The exposure distribution data is generated on the basis of the input data as first output data from the first trained function. The generated exposure distribution data may then be provided in a further step.

The provided exposure distribution data may for example be estimated by the trained function without there being any need for a measurement to detect the exposure distribution to be performed for this purpose. It is accordingly advantageously possible to dispense with such a measurement method.

For example, the exposure distribution data may be stored or at least buffered on an appropriate storage medium in the form of an appropriate computer-readable file, for example a two-dimensional image file, comprising the two-dimensional intensity distribution. The exposure distribution data may, for example, be provided by saving the computer-readable file on the storage medium.

This data may, for example, be used for documentation, for example in an electronic patient record. Provision may also be made for the exposure distribution data to be provided to an electronic system with a computing unit that may further process this data.

Unless otherwise stated, all the steps of the computer-implemented method may be carried out by a data processing device that has at least one computing unit. For example, the at least one computing unit is configured or adapted for carrying out the steps of the computer-implemented method. The at least one computing unit may to this end store for example one or more computer programs that, on execution, cause the at least one computing unit to carry out the computer-implemented method.

Improved exposure distribution data may advantageously be provided by the method. For example, the improved data does not underestimate local exposure peaks that may for example act at local points on a patient's skin. For example, the improved data may attribute skin reddening, that may occur on a local point of the irradiated area even weeks after radiation treatment, to the exposure peaks. Furthermore, if exposure peaks at local points of the irradiated area are known about, radiation treatment may be adapted such that any consequences, such as skin reddening for example, may be reduced.

The setting data, also designated imaging system settings, may for example be individually set by an imaging system user, for example by an attending physician or administrator. For example, this setting data may be stored or at least buffered on an appropriate storage medium in the form of an appropriate computer-readable file including the setting data. The setting data may be received, for example, by reading out the computer-readable file from the storage medium.

The exposure parameter is a variable or value in dosimetry and the basis for calculating the radiation exposure for an irradiated body or irradiated area during irradiation with the radiation source, for example during an X-ray capture with an X-ray machine, such as for example a transillumination, an angiography, or the like. The exposure parameter corresponds to the sum of the radiation exposure impinging on the irradiated area in the beam path from the radiation source. The unit of measure for the exposure parameter value is cGy×cm² or Gy×m². The radiation exposure parameter may for example correspond to a dose area product (DAP).

In X-ray-based imaging systems, such as X-ray-based angiography systems, the dose area product may to this end, for example, be measured as the exposure parameter in the imaging system's beam path using a measurement device known as a DAP chamber that sums the radiation exposure over an irradiated area as a single value.

The exposure parameter is for example dependent on the particular imaging system and its setting data. If the setting data changes, so too does the exposure parameter as a consequence. In this respect, the received exposure parameter corresponds to that which has been measured or determined in an alternative manner in the imaging system set with the received setting data. For example, this exposure parameter may be stored or at least buffered on an appropriate storage medium in the form of an appropriate computer-readable file including the exposure parameter. The exposure parameter may be received, for example, by reading out the computer-readable file from the storage medium.

The received intensity distribution corresponds to that which has been measured or determined at the radiation detector in an alternative manner, i.e., not by measurement, on the imaging system set with the received setting data at the radiation detector.

The intensity distribution data may correspond to a detector image of the radiation detector that depicts, for example graphically, the intensity distribution of the radiation on an irradiated detector area of the radiation detector. For example, this intensity distribution may be stored or at least buffered on an appropriate storage medium in the form of an appropriate computer-readable file, for example a two-dimensional image file, including the two-dimensional intensity distribution. The intensity distribution may be received, for example, by reading out the computer-readable file from the storage medium.

In an embodiment, the intensity distribution may be provided by the radiation detector being irradiated with the radiation from the radiation source set with the setting data of the imaging system. No object or a homogeneous object is located in the beam path, and wherein the radiation detector provides the intensity distribution as a two-dimensional detector image. A homogeneous object may be taken to be an object that has no influence on intensity distribution. Acrylic sheet of an appropriate thickness may, for example, be used for this purpose.

The setting data, the exposure parameter and the intensity distribution data may now be used as input data for the first trained function. The first trained function may for example be based on an artificial neural network (ANN), for example having at least one convolutional layer. For example, the trained function may be based on a generic inverse algorithm. For example, a computing unit may be configured to apply the trained function in order to generate the exposure data as first output data.

An ANN may be taken to be software code or a compilation of a plurality of software code components, the software code possibly including a plurality of software modules for different functions, for example one or more encoder modules and one or more decoder modules.

An ANN may be taken to be a nonlinear model or an algorithm that maps an input onto an output, the input being provided by an input feature vector or an input sequence and the output possibly being an output category for a classification task or a predicted sequence.

The ANN may for example be provided in computer-readable form.

The neural network may include a plurality of modules including an encoding module and a decoding module. These modules may be taken to be software modules or corresponding parts of the neural network. A software module may be taken to be software code that is functionally connected to and combined with a unit. A software module may include or implement a plurality of processing steps and/or data structures.

The modules may for example themselves constitute neural networks or subnetworks. Unless otherwise stated, a module of the neural network may be taken to be a trainable and for example trained module of the neural network. For example, the neural network and thus all its trainable modules may be fully trained before the method is carried out. In other implementations, however, different modules may be individually trained or pretrained. In other words, the method may correspond to an operational phase of the first trained function, for example of the ANN.

The first trained function may for example be trained such that it is suitable for generating realistic and meaningful exposure distribution data as output data. The first trained function may for example be trained and provided by way of a computer-implemented training method for training a first function.

The exposure distribution may be predicted extremely accurately and individually by using the first trained function. Furthermore, using the first trained function may enable the computing unit to generate the exposure distribution data extremely rapidly, robustly and using relatively little computing power.

The first trained function may for example be provided as a general function for a series of structurally identical or at least similar imaging systems or be applied to each specific imaging system from this series.

The method may for example be used during irradiation of a body by way of the imaging system, for example during a medical intervention, but also in materials testing.

Embodiments provide that the intensity distribution data is provided by a second trained function being applied to the setting data as second input data for the second trained function, the intensity distribution data being generated as second output data from the second function.

This is advantageous at least in that, for the purpose of providing the intensity distribution, no actual irradiation or no actual measurement need be performed for determining the intensity distribution on the basis of the setting data. Instead, the intensity distribution may be provided in a purely computer-implemented manner.

For example, the second output data from the second trained function may form part of the first input data for the first trained function. To this end, the second trained function provides the intensity distribution as a two-dimensional digital image, for example as a corresponding image file.

The second trained function may for example be provided as a specific function for an individual, specific imaging system from a series of structurally identical or at least similar imaging systems or be applied solely to the specific imaging system from this series. By linking the first trained function, that may be generally applied to the series of imaging systems, to the second trained function, the first trained function may, as it were, be specified for the specific imaging system.

For example, the first trained and second trained functions are combined with one another during use, for example during a medical intervention with a patient in the beam. This results in a further advantage to the effect that, when this combination is used in practice, the computing unit only has to receive the setting data and the exposure parameter in order to be able to generate the exposure distribution. This significantly speeds up and simplifies the method.

The second trained function may for example be trained such that it is suitable for generating realistic and meaningful intensity distribution data as output data. The second trained function may for example be trained and provided by way of a computer-implemented training method for training a second function. The second trained function may for example be based on an artificial neural network (ANN), for example having at least one convolutional layer. For example, the second trained function may be based on a generic inverse algorithm. For example, a computing unit may be configured to apply the second trained function in order to generate the intensity distribution as second output data.

At least one embodiment provides that the second trained function is provided by a second computer-implemented training method. The second computer-implemented training method for example has the steps of: receiving second input training data including setting data of the imaging system; receiving second output training data including intensity distribution data, the second output training data being related to the second input training data; training a second function on the basis of the second input training data and the second output training data by second output data being generated by application of the second function to the second input training data and by parameters of the second function being adapted on the basis of a comparison of the second output data with the second output training data; and providing the second trained function.

At least one embodiment provides that the exposure parameter is provided by a third trained function being applied to the setting data as third input data for the third function, the exposure parameter being generated as third output data from the third function.

This is advantageous at least in that, for the purpose of providing the exposure parameter, no actual irradiation or no actual measurement need be performed for determining the exposure parameter on the basis of the setting data. Instead, the exposure parameter may be provided in a purely computer-implemented manner.

For example, the third output data from the third trained function may form part of the first input data for the first trained function. To this end, the third trained function provides the exposure parameter as a value.

The third trained function may for example be provided as a specific function for an individual, specific imaging system from a series of structurally identical or at least similar imaging systems or be applied solely to the specific imaging system from this series.

A further advantage is thus obtained from a combination of the third trained function with the second trained function and the first trained function. When this combination is used, the computing unit only has to receive the setting data in order to be able to generate the exposure distribution. This considerably speeds up and simplifies the method.

The third trained function may for example be trained such that it is suitable for generating realistic and meaningful exposure parameters as output data. The third trained function may for example be trained and provided by way of a computer-implemented training method for training a third function. The third trained function may for example be based on an artificial neural network (ANN), for example having at least one convolutional layer. For example, the third trained function may be based on a generic inverse algorithm. For example, a computing unit may be configured to apply the third trained function in order to generate the exposure parameter as third output data.

At least one embodiment provides that the third trained function is provided by a third computer-implemented training method. The third computer-implemented training method for example has the steps of: receiving third input training data including setting data of the imaging system, receiving third output training data including exposure parameters, the third output training data being related to the third input training data; training a third function on the basis of the third input training data and the third output training data by third output data being generated by application of the third function to the third input training data and by parameters of the third function being adapted on the basis of a comparison of the third output data with the third output training data; and providing the third trained function.

At least one embodiment provides that the exposure parameter is a dose area product or a variable dependent on the dose area product.

The radiation source is in this case for example an X-ray tube. Similar variables are, however, also obtained for other types of ionizing radiation.

At least one embodiment provides that the intensity distribution data is a two-dimensional X-ray projection image. The intensity distribution may, for example, be represented with the assistance of the color intensity of the individual pixels of the X-ray projection image.

The X-ray projection image, also designated detector image, may for example be a graphical representation of a two-dimensional array of pixels, the pixel values possibly representing measured values of individual radiation sensors of the radiation detector.

At least one embodiment provides that the exposure distribution data is a two-dimensional image of the exposure distribution at an interventional reference point. The interventional reference point may here correspond to that location at which it is intended to place the body to be irradiated in the beam path. The exposure distribution may thus indicate precisely the distribution that acts or would act on the body to be irradiated.

At least one embodiment provides that the setting data of the imaging system includes at least operating parameters of the radiation source and/or of an imaging system collimator.

If, for example, the radiation source is an X-ray source, i.e., for example an X-ray tube, the operating parameters of the radiation source may for example include a peak kilovoltage (kVp), i.e., a maximum tube voltage that is applied to the X-ray tube when generating the X-rays as ionizing radiation. The radiation source operating parameters may also include a tube current of the X-ray tube and/or a focal spot size and so on.

A collimator is a physical barrier that focuses or parallelizes the high-energy electromagnetic waves such as X-rays or gamma radiation. For example, the spatial boundary of the radiation source beam may be stated or set with the assistance of the collimator operating parameters.

At least one embodiment provides that the imaging system is an X-ray-based imaging system and the radiation source an X-ray source, for example an X-ray tube.

A further aspect relates to a first computer-implemented training method for providing a first trained function that generates data relating to an exposure distribution for radiation from a radiation source of an imaging system. This method may for example be performed by a computing unit and for example includes the following steps.

In a first step of the method, first input training data is received. This includes setting data of the imaging system, together with radiation exposure parameters associated with the setting data, as well as data associated with the setting data and relating to an intensity distribution of the radiation on a radiation detector of the imaging system.

In a second step of the method, first output training data that includes exposure distribution data is received. The output training data is related to the input training data.

In a third step, a first function is trained on the basis of the first input training data and the first output training data by first output data being generated by application of the first function to the first input training data and by parameters of the first function being adapted on the basis of a comparison of the first output data with the first output training data.

In a fourth step, the first trained function is provided.

The input training data may for example be received using a first training interface of an electronic training system, and the output training data may be received by a second training interface of the electronic training system. The first trained function may be provided using a third training interface of the electronic training system.

The first function may for example be based on an artificial neural network (ANN), for example having at least one convolutional layer. For example, the function may be based on a generic inverse algorithm. For example, the electronic training system may be configured to train the first function. The first function may for example be trained such that it is suitable as a first trained function for generating or predicting realistic and meaningful radiation exposure distribution data as output data.

Comparing the output data with the output training data may for example determine an error in the function. For example, a “cost function” may be calculated in order to quantify the error. The parameters of the function may, for example, be adapted using a known algorithm in such a way that the cost function is minimized, for example iteratively.

At least one embodiment of the first computer-implemented training method provides that the output training data is provided by dosimetric measurements and/or dosimetric simulations.

The dosimetric measurements as output training data should here be made using the corresponding associated setting data as input training data. A dosimetric measurement may for example be carried out using a radiochromic film. The film contains a dye that changes color on irradiation with ionizing radiation, such that the degree of irradiation, the beam profile and the radiation exposure distribution may be characterized. The film may be digitized, for example by scanning, and appropriately digitally post-processed such that a two-dimensional digital image is obtained.

Alternatively, the exposure distribution data may be provided as output training data by dosimetric simulation on the basis of the associated setting data, the exposure parameter and the intensity distribution. A Monte Carlo simulation may, for example, be carried out for this purpose.

The intensity distribution and/or the exposure parameter as input training data may likewise be provided on the basis of measurements and/or simulations and/or by way of a trained function.

At least one embodiment of the first computer-implemented training method provides that the first trained function is provided for a series of imaging systems.

The first trained function may for example be provided as a general function for a series of structurally identical or at least similar imaging systems or be applied to each specific imaging system from this series.

At least one embodiment of the computer-implemented method for providing exposure distribution data provides that the first trained function is provided by the first computer-implemented training method.

Embodiments provide a second computer-implemented training method for providing a second trained function that generates data relating to an intensity distribution of radiation on a radiation detector of an imaging system with a radiation source. This method may for example be performed by a computing unit and for example includes the following steps.

In a first step of the method, second input training data that includes setting data of the imaging system may be received.

In a second step of the method, second output training data that includes intensity distribution data may be received. The second output training data is here related to the second input training data.

In a third step, a second function is trained on the basis of the second input training data and the second output training data by second output data being generated by application of the second function to the second input training data and by parameters of the second function being adapted on the basis of a comparison of the second output data with the second output training data.

In a fourth step, the second trained function is provided.

The input training data may for example be received using a first training interface of an electronic training system, and the output training data may be received by a second training interface of the electronic training system. The second trained function may be provided using a third training interface of the electronic training system.

The second function may for example be based on an artificial neural network (ANN), for example having at least one convolutional layer. For example, the function may be based on a generic inverse algorithm. For example, the electronic training system may be configured to train the second function. The second function may for example be trained such that it is suitable as a second trained function for generating or predicting realistic and meaningful radiation intensity distribution data as output data.

Comparing the output data with the output training data may for example determine an error in the function. For example, a “cost function” may be calculated in order to quantify the error. The parameters of the function may, for example, be adapted using a known algorithm in such a way that the cost function is minimized, for example iteratively.

At least one embodiment of the second computer-implemented training method provides that the second trained function is specifically provided for a specific imaging system from a series of imaging systems. The series of imaging systems may for example include structurally identical or at least structurally similar imaging systems.

The second output training data for training the second specific function may for example be provided by way of the associated specific system. For example, the intensity distribution data of the specific system may be obtained as second output training data by measurements using the specific system, wherein no object or a homogeneous object is located in the beam path. For example, such measurements may be or have been provided by regular, for example annual, servicing measurements.

For example, when it comes to training the second function, it may be sufficient to train it with a relatively small volume of training data. This may for example be because, when the second trained function is applied in the computer-implemented method for providing exposure distribution data, it is linked with the first trained function. The second trained function here merely serves to specify the first trained function for the specific system of the associated, second trained function, the first trained function having been trained with a relatively large volume of training data. In this combination, the relatively small volume of training data is consequently sufficient for training the second function.

At least one embodiment of the computer-implemented method for providing exposure distribution data provides that the second trained function is provided by the second computer-implemented training method.

A further aspect relates to a third computer-implemented training method for providing a third trained function, which data generates an exposure parameter for radiation from an imaging method radiation source. This method may for example be performed by a computing unit and for example includes the following steps.

In a first step of the method, third input training data that includes setting data of the imaging system may be received.

In a second step of the method, second output training data that includes exposure parameters may be received. The third output training data is here related to the third input training data.

In a third step, a third function is trained on the basis of the third input training data and the third output training data by third output data being generated by application of the third function to the third input training data and by parameters of the third function being adapted on the basis of a comparison of the third output data with the third output training data.

In a fourth step, the third trained function is provided.

The input training data may for example be received using a first training interface of an electronic training system, and the output training data may be received by a second training interface of the electronic training system. The third trained function may be provided using a third training interface of the electronic training system.

The third function may for example be based on an artificial neural network (ANN), for example having at least one convolutional layer. For example, the function may be based on a generic inverse algorithm. For example, the electronic training system may be configured to train the second function. The second function may for example be trained such that it is suitable as a second trained function for generating or predicting realistic and meaningful radiation intensity distribution data as output data.

Comparing the output data with the output training data may for example determine an error in the function. For example, a “cost function” may be calculated in order to quantify the error. The parameters of the function may, for example, be adapted using a known algorithm in such a way that the cost function is minimized, for example iteratively.

At least one embodiment of the third computer-implemented training method provides that the third trained function is specifically provided for a specific imaging system from a series of imaging systems. The series of imaging systems may for example include structurally identical or at least structurally similar imaging systems.

The third output training data for training the third specific function may for example be provided by way of the associated specific system. For example, the exposure parameters of the specific system may be obtained as third output training data by measurements using the specific system.

At least one embodiment of the computer-implemented method for providing exposure distribution data provides that the third trained function is provided by the third computer-implemented training method.

A further aspect relates to a data processing device with at least one computing unit that is adapted for carrying out a computer-implemented method for providing exposure distribution data for radiation from a radiation source of an imaging system.

For example, the data processing device for providing exposure distribution data for radiation from a radiation source of an imaging system is provided and has: a first interface for receiving setting data of the imaging system; a second interface for receiving a radiation exposure parameter associated with the setting data; a third interface for receiving data associated with the setting data and relating to an intensity distribution of the radiation on a radiation detector of the imaging system; a computing unit for applying a first trained function to the setting data, the exposure parameter and the intensity distribution data as first input data for the first function, the exposure distribution data being generated as first output data from the first function; and a fourth interface for providing the exposure distribution data.

A computing unit may for example be taken to mean a data processing device that contains a processing circuit. The computing unit may thus for example process data for carrying out computing operations. These may optionally also include operations for carrying out indexed access to a data structure, for example a look-up table (LUT).

The computing unit may for example contain one or more computers, one or more microcontrollers and/or one or more integrated circuits, for example one or more application-specific integrated circuits (ASIC), one or more field-programmable gate-arrays (FPGA), and/or one or more systems on a chip (SoC). The computing unit may also contain one or more processors, for example one or more microprocessors, one or more central processing units (CPU), one or more graphics processing units (GPU) and/or one or more signal processors, for example one or more digital signal processors (DSP). The computing unit may also contain a physical or virtual cluster of computers or others of the stated units.

In various embodiments, the computing unit contains one or more hardware and/or software interfaces and/or one or more memory units.

A memory unit may be configured as a volatile data memory, for example as a dynamic random access memory (DRAM) or static random access memory (SRAM), or as a nonvolatile data memory, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM) or as a phase-change random access memory (PCRAM).

Further embodiments of the data processing device follow directly from the various configurations of the associated method and vice versa. For example, individual features and corresponding explanations and advantages with regard to the various embodiments of the method may be applied mutatis mutandis to corresponding embodiments of the data processing device.

A further aspect provides a computer program with commands. If the commands are executed by at least one data processing device, for example a data processing device, the commands cause the data processing device to carry out the method for providing exposure distribution data for radiation from a radiation source of an imaging system.

The commands may, for example, take the form of program code. The program code may be provided, for example, as binary code or an assembler and/or as source code of a programming language, for example C, and/or as a program script, for example Python.

A further aspect relates to an electronically readable data storage medium including commands that, when the data storage medium is executed on at least one data processing device, for example on a data processing device, cause the data processing device to carry out the method for providing exposure distribution data for radiation from a radiation source of an imaging system.

The computer program and the computer-readable storage medium are in each case computer program products with the commands.

A further aspect provides an imaging system. The imaging system includes a data processing device, a radiation source for generating for example ionizing radiation, and a radiation detector for detecting parts of the radiation, for example parts of the ionizing radiation that have passed through an object to be imaged.

A further aspect relates to a first electronic training system for providing a first trained function that generates data relating to an exposure distribution for radiation from a radiation source of an imaging system including: a first training interface for receiving first input training data including setting data of the imaging system, radiation exposure parameters associated with the setting data, and data associated with the setting data and relating to an intensity distribution of the radiation on a radiation detector of the imaging system dependent on the setting data; a second training interface for receiving first output training data including exposure distribution data, the output training data being related to the input training data; a computing unit for training a first function on the basis of the first input training data and the first output training data by first output data being generated by application of the first function to the first input training data and by parameters of the first function being adapted on the basis of a comparison of the first output data with the first output training data; and a third training interface for providing the first trained function.

The first electronic training system is for example configured to carry out the first computer-implemented training method.

Further embodiments of the training system follow directly from the various configurations of the training method and vice versa. For example, individual features and corresponding explanations and advantages with regard to the various embodiments of the training method may be applied mutatis mutandis to corresponding embodiments of the training system.

A further aspect relates to a second electronic training system for providing a second trained function that generates data relating to an intensity distribution of radiation on a radiation detector of an imaging system with a radiation source: a fourth training interface for receiving second input training data including setting data of the imaging system; a fifth training interface for receiving second output training data including intensity distribution data, the output training data being related to the input training data; a computing unit for training a second function on the basis of the second input training data and the second output training data by second output data being generated by application of the second function to the second input training data and by parameters of the second function being adapted on the basis of a comparison of the second output data with the second output training data; and a sixth training interface for providing the second trained function.

The second electronic training system is for example configured to carry out the second computer-implemented training method.

Further embodiments of the training system follow directly from the various configurations of the training method and vice versa. For example, individual features and corresponding explanations and advantages with regard to the various embodiments of the training method may be applied mutatis mutandis to corresponding embodiments of the training system.

A further aspect relates to a third electronic training system for providing a third trained function that generates an exposure parameter for radiation from a radiation source of an imaging system including: a seventh training interface for receiving third input training data including setting data of the imaging system, an eighth training interface for receiving first output training data including exposure parameters, the output training data being related to the input training data; a computing unit for training a third function on the basis of the third input training data and the third output training data by third output data being generated by application of the third function to the third input training data and by parameters of the third function being adapted on the basis of a comparison of the third output data with the third output training data; and a ninth training interface for providing the third trained function.

The third electronic training system is for example configured to carry out the third computer-implemented training method.

Further embodiments of the training system follow directly from the various configurations of the training method and vice versa. For example, individual features and corresponding explanations and advantages with regard to the various embodiments of the training method may be applied mutatis mutandis to corresponding embodiments of the training system.

Further aspects relate to computer training programs including commands that, when the respective computer program is executed on an electronic training system, cause the electronic training system to carry out a respective training method.

Further aspects relate to electronically readable training data storage media including commands that, when the respective data storage medium is executed on an electronic training system, cause the electronic training system to carry out a respective training method.

For application cases or application situations that may arise during the described method or training method and are not described explicitly here, provision may be made for an error message and/or a request to input user feedback to be output and/or a default setting and/or a predetermined initial state to be set, in accordance with the method.

### BRIEF DESCRIPTION OF THE FIGURES

FIG. 1 depicts a schematic representation of an embodiment of an imaging system.

FIG. 2 depicts a schematic flowchart of an embodiment of a computer-implemented method for providing exposure distribution data.

FIG. 3 depicts is a schematic representation of an embodiment of the computer-implemented method for providing exposure distribution data.

FIG. 4 depicts a schematic flowchart of an embodiment of a first computer-implemented training method for providing a first trained function.

FIG. 5 depicts a schematic flowchart of an embodiment of a second computer-implemented training method for providing a second trained function.

FIG. 6 depicts a schematic flowchart of an embodiment of a third computer-implemented training method for providing a third trained function.

FIG. 7 depicts a schematic structure of an embodiment of a first trained function.

FIG. 8 depicts a schematic structure of an embodiment of a second trained function;

FIG. 9 depicts a schematic representation of a first embodiment of an artificial neural network (ANN) of a trained function.

FIG. 10 depicts a schematic representation of a first embodiment of a convolutional ANN of a trained function.

### DETAILED DESCRIPTION

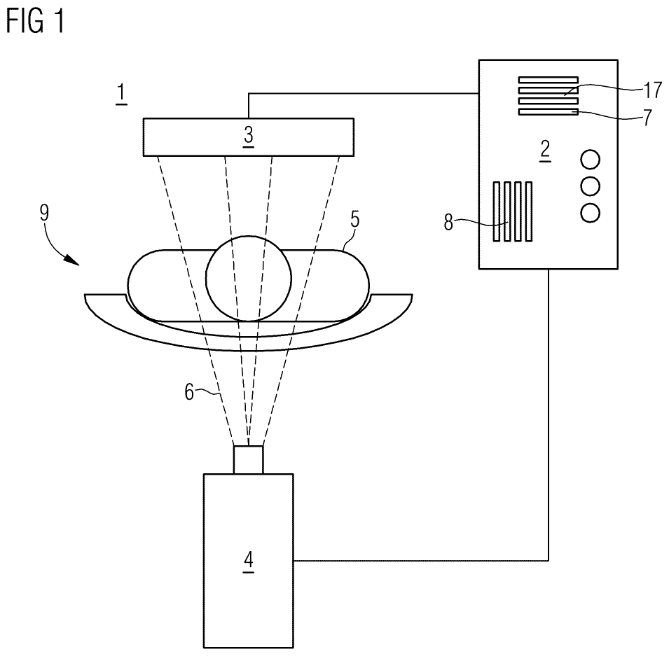

FIG. 1 is a schematic representation of an embodiment of an imaging system 1. The imaging system 1 includes at least one data processing device 2 and an imaging modality that has a radiation source 4 for generating ionizing radiation 6 and a radiation detector 3 for detecting parts of the ionizing radiation 6. For example, the imaging modality is configured as an X-ray-based imaging modality, for instance as an X-ray-based angiography system, and has an X-ray source as the radiation source 4 and an X-ray detector as the radiation detector 3. A patient is furthermore shown as the object 5 to be imaged that is located at an interventional reference point 9 of the imaging system 1.

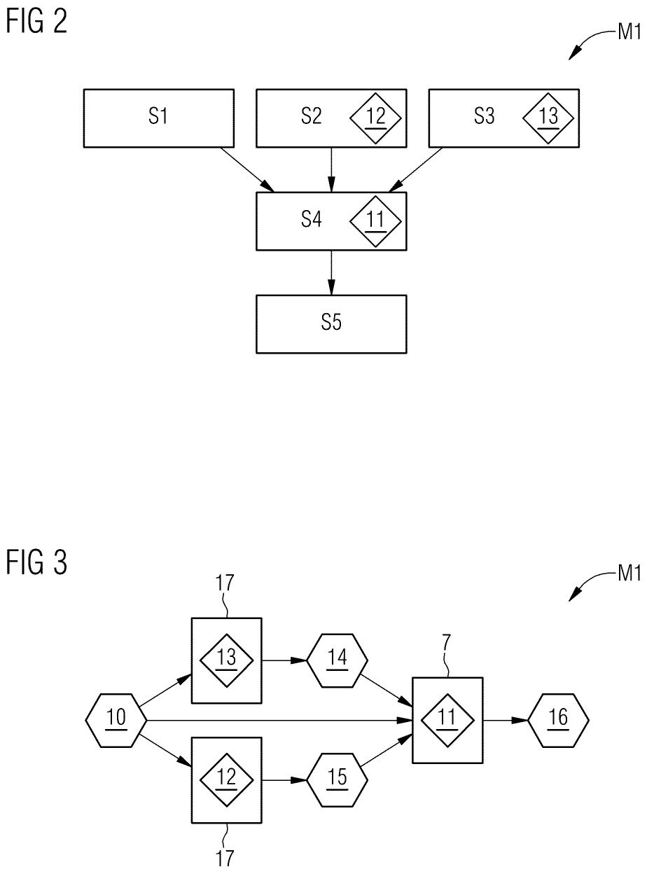

The data processing device 2 has at least one computing unit 7, 17. The at least one computing unit 7 is set up to carry out a computer-implemented method M1 for providing exposure distribution data for radiation 6 from the radiation source 4 of the imaging system 1. FIG. 2 depicts a schematic flowchart of an embodiment of such a method M1. A corresponding computer program product 8 may to this end be executed on the data processing device 2.

FIG. 2 depicts a schematic flowchart and FIG. 3 a schematic representation of an embodiment of a computer-implemented method M1 for providing exposure distribution 16 data that may for example be carried out by the computing unit 7, for example the data processing device 2. FIG. 2 and FIG. 3 are discussed together below.

In a first step S1 of the method M1, setting data 10 of the imaging system 1 may be received by the computing unit 7. In one embodiment, this setting data 10 of the imaging system 1 may include at least operating parameters of the radiation source 4 and/or a collimator of the imaging system 1.

In a second step S2 of method M1, the data associated with the setting data 10 and relating to an intensity distribution 15 of the radiation 6 on a radiation detector 3 of the imaging system 1 may be received by the computing unit 7. The intensity distribution 15 data may, for example, be a detector image, for example a two-dimensional X-ray projection image.

The intensity distribution 15 data may for example be provided by a second trained function 12 being applied to the setting data 10 as second input data for the second trained function 12, the intensity distribution 15 data being generated as second output data from the second trained function 12. The second trained function 12 may for example be applied by the further computing unit 17, the further computing unit 17 and the computing unit 7 being configurable as a common computing unit 7.

In a third step S3 of method M1, an exposure parameter 14 of the radiation 6 and associated with the setting data 10 may be received by the computing unit 7. The exposure parameter 14 may, for example, be a dose area product (DAP) or a variable dependent on the dose area product.

The exposure parameter 14 may for example be provided by a third trained function 13 being applied to the setting data 10 as third input data for the third trained function 13, the exposure parameter 14 being generated as third output data from the third trained function 13. The third trained function 13 may for example be applied by the further computing unit 17, the further computing unit 17 and the computing unit 7 being configurable as a common computing unit 7.

Alternatively, the exposure parameter may be measured using a DAP chamber in the beam path of the imaging system.

In a fourth step S4 of method M1, a first trained function 11 may be applied to the setting data 10, the exposure parameter 14 and the intensity distribution 15 data as first input data for the first trained function 11, the exposure distribution 16 data being generated as first output data from the first trained function.

The exposure distribution 16 data may for example be a two-dimensional image of the exposure distribution 16 at the interventional reference point 9 of the imaging system 1.

In a fifth step S5, the exposure distribution 16 data is provided.

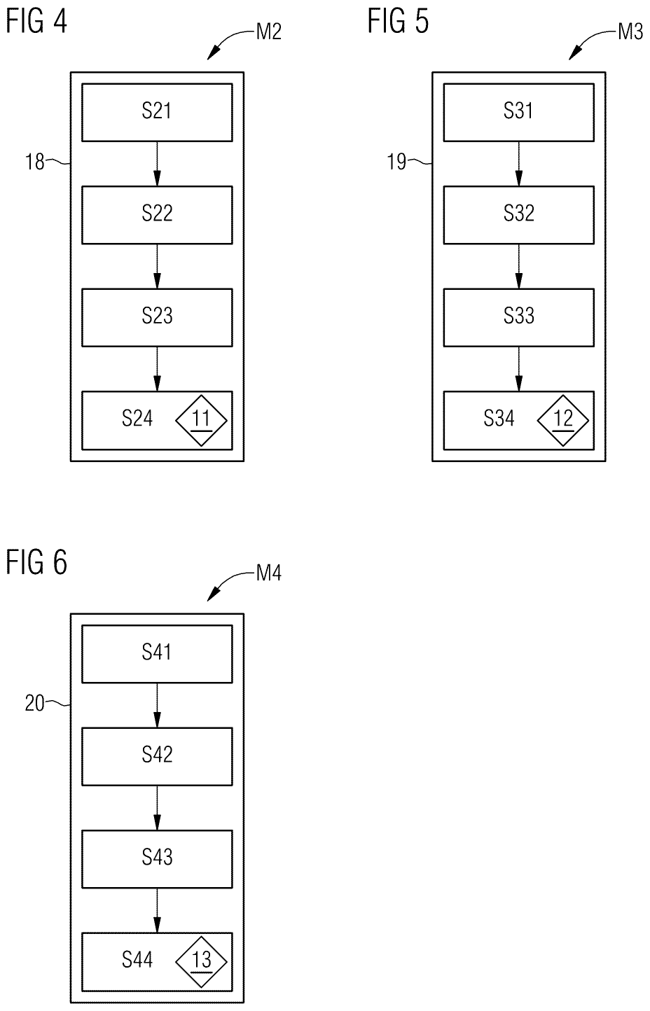

FIG. 4 depicts a schematic flowchart of an embodiment of a first computer-implemented training method M2 for providing a first trained function 11 that may be carried out, for example, by a first electronic training system 18 with at least one computing unit.

In a first step S21, first input training data is received that includes setting data 10′ of an imaging system 1, exposure parameters 14′ associated with the setting data 10′ and relating to the radiation 6, and data associated with the setting data 10′ and relating to an intensity distribution 15′ of the radiation 6 on a radiation detector 3 of the imaging system 1.

In a second step S22, first output training data including exposure distribution (16′) data is received, the first output training data being related to the first input training data.

In a third step S23, a first function is trained on the basis of the first input training data and the first output training data by first output data being generated by application of the first function to the first input training data and by parameters of the first function being adapted on the basis of a comparison of the first output data with the first output training data.

In a fourth step S24, the first trained function 11 is provided.

FIG. 5 depicts a schematic flowchart of an embodiment of a second computer-implemented training method M3 for providing a second trained function 12 that may be carried out, for example, by a second electronic training system 19 with at least one computing unit.

In a first step S31, second input training data that includes setting data 10′ of an imaging system 1 is received.

In a second step S32, second output training data including intensity distribution (15′) data is received, the second output training data being related to the second input training data.

In a third step S33, a second function is trained on the basis of the second input training data and the second output training data by second output data being generated by application of the second function to the second input training data and by parameters of the second function being adapted on the basis of a comparison of the second output data with the second output training data.

In a fourth step S34, the second trained function 12 is provided.

FIG. 6 depicts a schematic flowchart of an embodiment of a third computer-implemented training method M4 for providing a third trained function 13 that may be carried out, for example, by a third electronic training system 20 with at least one computing unit.

In a first step S41, third input training data that includes setting data 10′ of an imaging system 1 is received.

In a second step S42, third output training data including exposure parameters (14′) is received, the third output training data being related to the third input training data.

In a third step S43, a third function is trained on the basis of the third input training data and the third output training data by third output data being generated by application of the third function to the third input training data and by parameters of the third function being adapted on the basis of a comparison of the third output data with the third output training data.

In a fourth step S44, the third trained function 13 is provided.

FIG. 7 depicts a schematic structure of an embodiment of a first trained function 11. In this embodiment, the first trained function 11 may have at least one encoder 21 and one decoder 22.

For example, the intensity distribution 15 data, that is here shown for example as a graph in which the radiation intensity of a two-dimensional XY area is plotted on a z axis, is used as input data for the encoder, intensity distribution 15 data of variable length being encodable into a sequence of fixed length. The setting data 10 and the exposure parameter 14 for example already have a sequence of fixed length and may thus be used directly as input data for the decoder 22. The decoder 22 is then configured to generate, on the basis of the input data, the exposure distribution 16 data, that is here shown for example as a graph in which the exposure intensity of a two-dimensional XY area is plotted on a z axis.

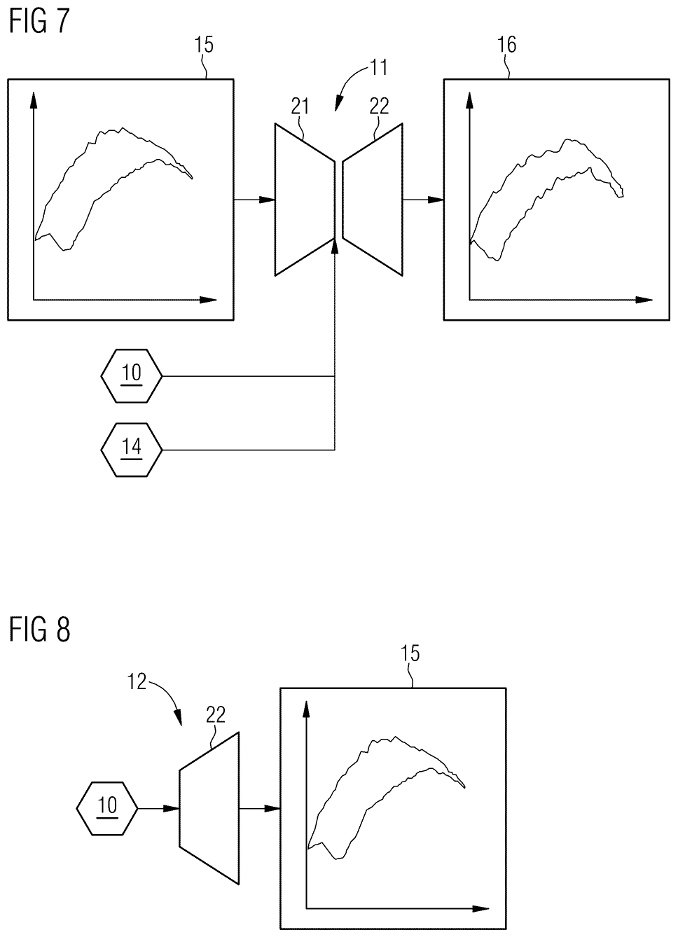

FIG. 8 depicts a schematic structure of an embodiment of a second trained function 12. In this embodiment, the second trained function 12 may have at least one decoder 22. The setting data 10 for example already has a sequence of fixed length and may thus be used directly as input data for the decoder 22. The decoder 22 is then configured to generate, on the basis of the input data, the intensity distribution 15 data, that is here shown for example as a graph in which the radiation intensity of a two-dimensional XY area is plotted on a z axis.

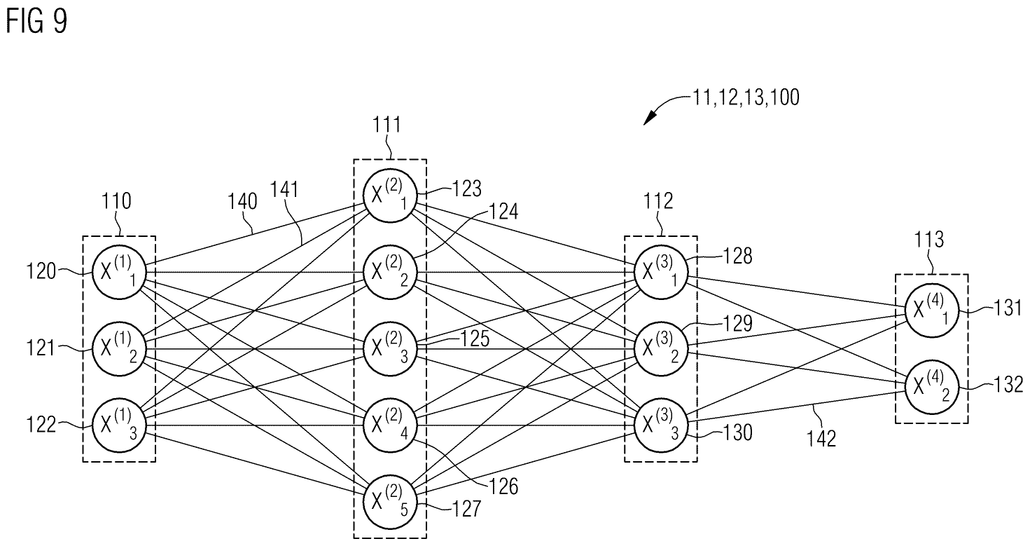

FIG. 9 depicts an embodiment of an artificial neural network (ANN) 100 for a function or for a trained function, for example for a first trained function 11 and/or a second trained function 12 and/or for a third trained function 13. Alternative terms for “artificial neural network” are “neural network”, “artificial neural network” or “neural net”.

The artificial neural network 100 includes nodes 120-132 and edges 140-142, each edge 140-142 being a directional connection from a first node 120-132 to a second node 120-132. In general, the first node 120-132 and the second node 120-132 are different nodes 120-132, but it is also possible for the first node 120-132 and second node 120-132 to be identical. In FIG. 6, the edge 140 is for example a directional connection from node 120 to node 123, and the edge 142 is a directional connection from node 130 to node 132. An edge 140-142 from a first node 120-132 to a second node 120-132 is also designated an “incoming edge” for the second node 120-132 and an “outgoing edge” for the first node 120-132.

In this embodiment, nodes 120-132 of the artificial neural network 100 may be arranged in layers 110-113, with the layers possibly having an intrinsic order that is introduced by edges 140-142 between nodes 120-132. For example, edges 140-142 may only be present between adjacent layers of nodes. In the embodiment shown, there is an input layer 110 that only includes nodes 120-122 without incoming edges, an output layer 113 that only includes nodes 131, 132 without outgoing edges, and concealed layers 111, 112 between input layer 110 and output layer 113. In general, the number of hidden layers 111, 112 may be selected at will. The number of nodes 120-122 within entry layer 110 generally relates to the number of entry values of the neural network, and the number of nodes 131, 132 within exit layer 113 generally relates to the number of exit values of the neural network.

For example, each node 120-132 of neural network 100 may be assigned a (real) number as its value. In this case, x(n)i designates the value of the i^{th} node 120-132 of the n^{th} layer 110-113. The values of nodes 120-122 of the entry layer 110 correspond to the entry values of neural network 100, while the values of nodes 131, 132 of exit layer 113 correspond to the exit value of the neural network 100. Each edge 140-142 may furthermore have a weight that is a real number, for example the weight is a real number within the range [−1, 1] or within the range [0, 1]. In this case, w(m,n)i,j designates the weight of the edge between the ith node 120-132 of the m^{th} layer 110-113 and the j^{th} node 120-132 of the nth layer 110-113. The abbreviation w(n)i,j is moreover defined for the weight w(n,n+1)i,j.

The exit values of the neural network 100 are calculated for example by propagating the entry values through the neural network. For example, the values of nodes 120-132 of the (n+1)^{th} layer 110-113 are calculated on the basis of the values of nodes 120-132 of the nth layer 110-113 by

$${x}_{j}^{( n + 1 )} = f ( {\sum }_{i} {x}_{i}^{( n )} \cdot {w}_{i , j}^{( n )} ) .$$

The function f is here a transfer function (another term is “activation function”). Known transfer functions are step functions, sigmoid functions (e.g., the logistic function, the generalized logistic function, the hyperbolic tangent, the arctangent function, the error function, the smoothstep function), or rectifier functions. The transfer function is primarily used for normalization.

The values are for example propagated layer-by-layer through the neural network, the values of entry layer 110 being provided by the input of neural network 100, the values of the first concealed layer 111 being calculable on the basis of the values of entry layer 110 of the neural network, the values of the second concealed layer 112 being calculable on the basis of the values of the first concealed layer 111, etc.

The neural network 100 has to be trained with training data in order to define the values w(m,n)i,j for the edges. The training data for example includes training input data and training output data (designated ti). In a training step, the neural network 100 is applied to the training input data in order to generate calculated output data. For example, the training data and the calculated output data include a number of values that corresponds to the number of nodes of the output layer.

For example, a comparison between the calculated output data and the training data is used to recursively adapt the weights within the neural network 100 (backpropagation algorithm). For example, the weights are modified according to

$${{w}^{′}}_{i , j}^{( n )} = {w}_{i , j}^{( n )} - γ \cdot {δ}_{j}^{( n )} \cdot {x}_{i}^{( n )} ,$$

wherein γ is a learning rate, and the numbers δ(n)j may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

on the basis of δ⁽ⁿ⁺¹⁾ⱼ, if the (n+1)^{th} layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)^{th} layer is the exit layer 113, wherein f′ is the first derivative of the activation function and y(n+1)j the comparison training value for the j^{th} node of exit layer 113.

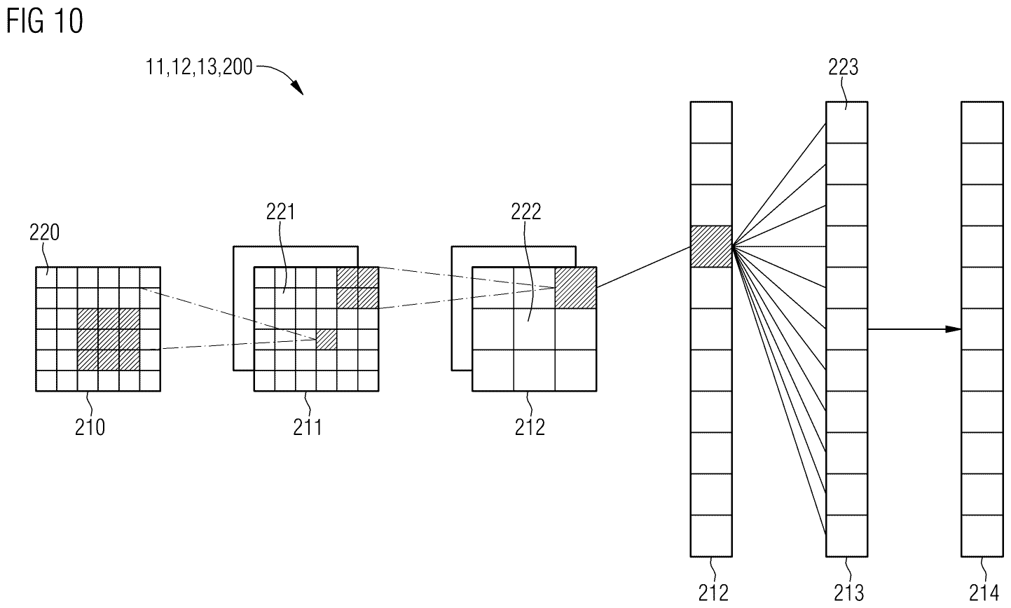

FIG. 10 depicts an embodiment of a convolutional neural network 200 for the function or trained function. In the embodiment shown, the convolutional neural network 200 includes an input layer 210, a convolutional layer 211, a pooling layer 212, a fully connected layer 213 and an output layer 214. The convolutional neural network 200 may alternatively also include a plurality of convolutional layers 211, a plurality of pooling layers 212 and a plurality of fully linked layers 213 together with other kinds of layers. The order of the layers may be selected at will; fully linked layers 213 are generally used as the final layers before the output layer 214.

For example, in a convolutional neural network 200, the nodes 220-224 of a layer 210-214 may be regarded as a d-dimensional matrix or as a d-dimensional image. For example, in the two-dimensional case the value of the node 220-224 indicated with i and j in the n^{th} layer 210-214 may be designated x(n)[i,j]. However, the arrangement of nodes 220-224 of a layer 210-214 has no effect on the calculations as such carried out in the convolutional neural network 200, as these result solely from the structure and weights of the edges.

For example, a convolutional layer 211 is characterized by the structure and weights of the incoming edges that form a convolution operation on the basis of a specific number of cores. For example, the structure and weights of the incoming edges are selected such that the values x⁽ⁿ⁾ₖ of nodes 221 of convolutional layer 211 are calculated as a convolution x⁽ⁿ⁾ₖ=Kₖ*x⁽ⁿ⁻¹⁾ on the basis of the values x⁽ⁿ⁻¹⁾ of nodes 220 of preceding layer 210, the convolution being defined, in the two-dimensional case, as

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

The k^{th} core Kₖ is here a d-dimensional matrix (in this case a two-dimensional matrix) that is generally small in comparison with the number of nodes 220-224 (e.g., a 3×3 matrix or a 5×5 matrix). This means for example that the weights of the incoming edges are not independent but are selected such that they provide the convolution equation. For example, for a kernel that is a 3×3 matrix, there are only 9 independent weights (each entry in the kernel matrix corresponds to an independent weight), irrespective of the number of nodes 220-224 in the respective layer 210-214. For example, for a convolutional layer 211, the number of nodes 221 in the convolutional layer is identical to the number of nodes 220 in the preceding layer 210 multiplied by the number of kernels.

If the nodes 220 of the preceding layer 210 are arranged as a d-dimensional matrix, using a plurality of cores may be interpreted as adding a further dimension (designated “depth” dimension), such that nodes 221 of convolutional layer 221 are arranged as a (d+1)-dimensional matrix. If nodes 220 of the preceding layer 210 are already arranged as a (d+1)-dimensional matrix with a depth dimension, using a plurality of cores may be interpreted as an extension along the depth dimension, such that nodes 221 of convolutional layer 221 are likewise arranged as a (d+1)-dimensional matrix, the size of the (d+1)-dimensional matrix in relation to the depth dimension being greater by a factor of the number of cores than in the preceding layer 210.

The advantage of using convolutional layers 211 is that a spatially local correlation of the input data may be utilized by a local connectivity pattern being enforced between nodes of adjacent layers, for example by each node only being connected to a small region of the nodes of the preceding layer.

In the embodiment shown, the entry layer 210 includes 36 nodes 220 that are arranged as a two-dimensional 6×6 matrix. The convolutional layer 211 includes 72 nodes 221 that are arranged as two two-dimensional 6×6 matrices, each of the two matrices being the result of convolving the values of the input layer with a kernel. Equivalently, the nodes 221 of the convolutional layer 211 may be interpreted as a three-dimensional 6×6×2 matrix, with the last dimension being the depth dimension.

A pooling layer 212 may be characterized by the structure and weights of the incoming edges and the activation function of their nodes 222 that form a pooling operation on the basis of a nonlinear pooling function f. In the two-dimensional case, the values x⁽ⁿ⁾ of nodes 222 of the pooling layer 212 may, for example, be calculated on the basis of the values x⁽ⁿ⁻¹⁾ of the nodes 221 of preceding layer 211 as follows:

$${x}^{( n )} [ i , j ] = f ( {x}^{( n - 1 )} [ {id}_{1} , {jd}_{2} ] , ... , {x}^{( n - 1 )} [ {id}_{1} + {d}_{1} - 1 , {jd}_{2} + {d}_{2} - 1 ] )$$

In other words, by using a pooling layer 212, the number of nodes 221, 222 may be reduced by a number d1,d2 of neighboring nodes 221 in the preceding layer 211 being replaced by a single node 222 that is calculated as a function of the values of the number of neighboring nodes in the pooling layer. The pooling function f may for example be the max function, the average or the L2 standard. For example, in a pooling layer 212, the weights of the incoming edges are fixed and are not modified by training.

The advantage of using a pooling layer 212 is that the number of nodes 221, 222 and the number of parameters is reduced. This results in a reduction in computational effort in the network and control of overadaptation.

In the embodiment shown, the pooling layer 212 is max pooling in which four neighboring nodes are replaced by just one node, the value being the maximum of the values of the four adjacent nodes. Max pooling is applied to each d-dimensional matrix of the preceding layer; in this embodiment, max pooling is applied to each of the two two-dimensional matrices, whereby the number of nodes is reduced from 72 to 18.

A fully connected layer 213 may be characterized in that a majority, for example all, of the edges between the nodes 222 of the preceding layer 212 and the nodes 223 of the fully connected layer 213 are present, the weight of each of the edges being individually settable.

In this embodiment, the nodes 222 of the preceding layer 212 of the fully linked layer 213 are represented both as two-dimensional matrices and additionally as non-contiguous nodes (shown as node lines, the number of nodes having been reduced for greater ease of depiction). In this embodiment, the number of nodes 223 in the fully connected layer 213 is identical to the number of nodes 222 in the preceding layer 212. Alternatively, nodes 222 and 223 may also differ in number.

Moreover, in this embodiment, the values of nodes 224 of the exit layer 214 are determined by applying the softmax function to the values of nodes 223 of the preceding layer 213. As a result of applying the softmax function, the sum of the values of all nodes 224 of the output layer is 1, and all values of all nodes 224 of the output layer are real numbers between 0 and 1. For example when the convolutional neural network 200 is used for categorizing input data, the values of the output layer may be interpreted as the probability of the input data falling into one of the various categories.

A convolutional neural network 200 may also contain a “rectified linear unit” (ReLU) layer. For example, the number of nodes and the structure of the nodes in a ReLU layer correspond to the number of nodes and the structure of the nodes in the preceding layer. For example, the value of each node in the ReLU layer is calculated by applying a rectification function to the value of the corresponding node in the preceding layer. Examples of rectification functions are f(x)=max(0,x), the tangential hyperbolic function or the sigmoid function.

Convolutional neural networks 200 may for example be trained on the basis of the backpropagation algorithm. Overadaptation may be prevented by using regularization methods, for example the omission of nodes 220-224, stochastic pooling, the use of artificial data, weight reduction on the basis of the L1 or L2 standard, or max standard limitations.

Overall, the examples show how a machine learning method may be provided for estimating an X-ray exposure distribution.

A non-homogeneous X-ray beam results in a non-homogeneous exposure distribution. The estimates of skin (organ, etc.) dose resulting therefrom are incorrect as the dose is locally higher/lower than that determined with the DAP chamber.

One important aspect of an embodiment is the prediction of a non-homogeneous beam (dose distribution) with the assistance of a machine learning model, for example a trained function that has been trained with the assistance of measured exposure and detector intensity distributions.

A non-homogeneous beam results in a non-homogeneous exposure distribution on the beam and a non-homogeneous intensity distribution on the detector.

One important aspect of an embodiment is therefore using the intensity distribution of a specific system's detector to “personalize” a general exposure distribution model.

One important aspect of an embodiment involves combining two machine learning models, for example the first trained function and the second trained function. The first trained function may forecast the dose distribution in the beam on the basis of the intensity distribution of the detector (with no or a homogeneous object in the beam), tube and collimator settings and the DAP (dose area product). This function may be trained with the assistance of dosimetry measurements (e.g., using films) or simulations (e.g., Monte Carlo simulations) of a plurality of systems and detector images as well as tube and collimator settings.

The second trained function predicts the system-specific detector intensity distribution (with no or a homogeneous object in the beam) with the assistance of the tube and collimator settings. This model may be trained with the assistance of data (detector images and tube and collimator settings) that have been collected, for example, during annual servicing measurements.

During use, for example during a medical intervention with a patient in the beam, the two models, i.e., the first trained function and the second trained function, may be combined, so enabling prediction of the dose distribution in the beam on the basis of the tube and collimator settings.

As a consequence, it is advantageously possible to ensure more accurate X-ray dose calculation. For example, it may be ensured that the local peak dose (skin dose) is not underestimated.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing exposure distribution data for radiation from a radiation source of an imaging system, the method comprising:
receiving setting data of the imaging system;
receiving a radiation exposure parameter associated with the setting data;
receiving data associated with the setting data and relating to an intensity distribution of the radiation on a radiation detector of the imaging system;
applying a first trained function to the setting data, the exposure parameter and the intensity distribution data as first input data for the first trained function, wherein the exposure distribution data is generated as first output data from the first trained function; and
providing the exposure distribution data;
wherein the intensity distribution data is provided by a second trained function being applied to the setting data as second input data for the second trained function, wherein the intensity distribution data is generated as second output data from the second trained function.

2. The computer-implemented method of claim 1, wherein the second trained function is provided by a second computer-implemented training method, the computer-implemented training method comprising:
receiving second input training data comprising setting data of the imaging system;
receiving second output training data comprising intensity distribution data, wherein the second output training data is related to the second input training data;
training a second function on a basis of the second input training data and the second output training data by second output data being generated by application of the second function to the second input training data and by parameters of the second function being adapted on a basis of a comparison of the second output data with the second output training data; and
providing the second trained function.

3. The computer-implemented method of claim 1, wherein the exposure parameter is provided by a third trained function being applied to the setting data as third input data for the third trained function, wherein the exposure parameter is generated as third output data from the third trained function.

4. The computer-implemented method of claim 3, wherein the third trained function is provided by a third computer-implemented training method, the third computer-implemented training method comprising:
receiving third input training data comprising setting data of the imaging system;
receiving third output training data comprising exposure parameters, wherein the third output training data is related to the third input training data;
training a third function on a basis of the third input training data and the third output training data by third output data being generated by application of the third function to the third input training data and by parameters of the third function being adapted on a basis of a comparison of the third output data with the third output training data; and
providing the third trained function.

5. The computer-implemented method of claim 1, wherein the exposure parameter is a dose area product or a variable dependent on the dose area product.

6. The computer-implemented method of claim 1, wherein the intensity distribution data is a two-dimensional X-ray projection image.

7. The computer-implemented method of claim 1, wherein the exposure distribution data is a two-dimensional image of the exposure distribution at an interventional reference point of the imaging system.

8. The computer-implemented method of claim 1, wherein the setting data of the imaging system includes at least operating parameters of the radiation source and/or a collimator of the imaging system.

9. The computer-implemented method of claim 1, wherein the imaging system is an X-ray-based imaging system and the radiation source is an X-ray source.

10. A computer-implemented training method for providing a first trained function that generates data relating to an exposure distribution for radiation from a radiation source of an imaging system, and for providing a trained second function that generates data relating to an intensity distribution for radiation from the radiation source of the imaging system, the method comprising:
receiving first input training data comprising: setting data of the imaging system, radiation exposure parameters associated with the setting data, and data associated with the setting data and relating to an intensity distribution of the radiation on a radiation detector of the imaging system;
receiving first output training data comprising exposure distribution data, wherein the first output training data is related to the first input training data;
training a first function on a basis of the first input training data and the first output training data by first output data being generated by application of the first function to the first input training data and by parameters of the first function being adapted on a basis of a comparison of the first output data with the first output training data;
providing the first trained function;
receiving second input training data comprising setting data of the imaging system;
receiving second output training data comprising intensity distribution data, wherein the second output training data is related to the second input training data;
training a second function on a basis of the second input training data and the second output training data by second output data being generated by application of the second function to the second input training data and by parameters of the second function being adapted on a basis of a comparison of the second output data with the second output training data; and
providing the second trained function.

11. The computer-implemented training method of claim 10, wherein the output training data is provided by dosimetric measurements and/or dosimetric simulations.

12. An imaging system for providing exposure distribution data for radiation from a radiation source of the imaging system, the imaging system comprising:
the radiation source for generating radiation;
a radiation detector for detecting parts of the radiation; and
a data processing device configured to:
receive setting data of the imaging system;
receive a radiation exposure parameter associated with the setting data;
receive data associated with the setting data and relating to an intensity distribution of the radiation on a radiation detector of the imaging system;
apply a first trained function to the setting data, the exposure parameter and the intensity distribution data as first input data for the first trained function, wherein the exposure distribution data is generated as first output data from the first trained function; and
provide the exposure distribution data;
wherein the intensity distribution data is provided by a second trained function being applied to the setting data as second input data for the second trained function, wherein the intensity distribution data is generated as second output data from the second trained function.

13. The imaging system of claim 12, wherein the second trained function is provided by a second computer-implemented training method, the computer-implemented training method comprising:
receiving second input training data comprising setting data of the imaging system;
receiving second output training data comprising intensity distribution data, wherein the second output training data is related to the second input training data;
training a second function on a basis of the second input training data and the second output training data by second output data being generated by application of the second function to the second input training data and by parameters of the second function being adapted on a basis of a comparison of the second output data with the second output training data; and
providing the second trained function.

14. The imaging system of claim 12, wherein the exposure parameter is provided by a third trained function being applied to the setting data as third input data for the third trained function, wherein the exposure parameter is generated as third output data from the third trained function.

15. The imaging system of claim 14, wherein the third trained function is provided by a third computer-implemented training method, the third computer-implemented training method comprising:
receiving third input training data comprising setting data of the imaging system;
receiving third output training data comprising exposure parameters, wherein the third output training data is related to the third input training data;
training a third function on a basis of the third input training data and the third output training data by third output data being generated by application of the third function to the third input training data and by parameters of the third function being adapted on a basis of a comparison of the third output data with the third output training data; and
providing the third trained function.

15. The imaging system of claim 12, wherein the exposure parameter is a dose area product or a variable dependent on the dose area product.

16. The imaging system of claim 12, wherein the intensity distribution data is a two-dimensional X-ray projection image.

17. The imaging system of claim 12, wherein the exposure distribution data is a two-dimensional image of the exposure distribution at an interventional reference point of the imaging system.

18. The imaging system of claim 12, wherein the setting data of the imaging system includes at least operating parameters of the radiation source and/or a collimator of the imaging system.

19. The imaging system of claim 12, wherein the imaging system is an X-ray-based imaging system and the radiation source is an X-ray source.
